(19) United States
(12) Patent Application Publication
(10) Pub. No.: US 2024/0208900 A1
(43) Pub. Date: Jun. 27, 2024

(54) SYNTHESIS OF DIMETHYL-C17-32-ALKYL SULFONIUM SALTS

(71) Applicant: HELM AG, Hamburg (DE)

(72) Inventors: Joerg STROOT, Hamburg (DE); Serhii ZHERSH, Kyiv (UA); Vasyl RIPENKO, Kyiv, Brovary (UA)

(73) Assignee: HELM AG, Hamburg (DE)

(21) Appl. No.: 18/286,904

(22) PCT Filed: Mar. 31, 2022
(86) PCT No.: PCT/EP2022/058566

§ 371 Date: Oct. 13, 2023

(30) Foreign Application Priority Data
Apr. 16, 2021 (EP) 21168807.2

(51) Int. Cl. C07C 381/12

(52) U.S. Cl. CPC C07C 381/12

(57) ABSTRACT

The invention relates to a process for the preparation of a dimethyl-C₁₇₋₃₂-alkyl sulfonium salt, preferably a dimethyl-C₁₇₋₃₂-alkyl sulfonium halide, more preferably a dimethyl-C₁₇₋₃₂-alkyl sulfonium chloride, still more preferably dimethyl octadecyl sulfonium chloride, the process comprising the steps of: providing a mixture of a C₁₇₋₃₂-alkyl halide and dimethyl sulfide; and reacting the C₁₇₋₃₂-alkyl halide with the dimethyl sulfide.

This application is a 371 of International Patent Application No. PCT/EP2022/058566, filed Mar. 31, 2022, which claims priority of European Patent Application No. 21 168 807.2, filed on Apr. 14, 2021, the contents of which patent applications are hereby incorporated herein by reference.

The invention relates to a process for the preparation of a dimethyl-C₁₇₋₃₂-alkyl sulfonium salt, preferably a dimethyl-C₁₇₋₃₂-alkyl sulfonium halide, more preferably a dimethyl-C₁₇₋₃₂-alkyl sulfonium chloride, still more preferably dimethyl octadecyl sulfonium chloride, the process comprising the steps of: providing a mixture of a C₁₇₋₃₂-alkyl halide and dimethyl sulfide; and reacting the C₁₇₋₃₂-alkyl halide with the dimethyl sulfide.

Trialkyl sulfonium halides (also referred to as trialkyl sulfanium halides) are known from the prior art, including dimethylalkyl sulfonium halides such as dimethyl(octadecyl)sulfonium chloride (CAS 2491668-67-6).

WO 2020/201698 A1 relates to antifungal compositions comprising single alkyl chain cationic antifungal compounds including octadecyl dimethyl sulfonium salts. The chemical synthesis of these compounds is not described.

Synthetic concepts for the synthesis of dimethyl-alkyl sulfonium salts may start from a fatty alcohol which is converted into the corresponding alkyl halide. The alkyl halide is then either directly reacted with dimethyl sulfide, or the alkyl halide is first reacted with methyl thiol thereby providing the methyl alkyl thioether as an intermediate, which is then subsequently methylated. Further synthetic concepts may directly convert the fatty alcohol with dimethyl sulfide. Still further synthetic concepts may convert the fatty alcohol with hydrogen sulfide into the corresponding alkyl thiol which is then subsequently methylated twice. Yet further synthetic concepts may start from an alpha-alkene which is converted with hydrogen sulfide into the corresponding alkyl thiol which is then subsequently methylated twice.

Methylating agents such as iodomethane are comparatively expensive making the overall synthesis at such comparatively poor yield not attractive for synthesis on industrial scale.

DE 709 420 relates to the synthesis of octadecyl dimethyl sulfonium methosulfate by reacting octadecyl mercaptan with dimethyl sulfate for several hours at 90 to 100° C. This synthetic concept has various disadvantages: The starting material octadecyl mercaptan is not easily accessible from octadecyl alcohol. Further, two equivalents of dimethyl sulfate are needed as methylating agent. In consequence, the reaction mixture contains one equivalent monomethyl sulfate (methyl sulfuric acid) as a byproduct which needs to be separated in a laborious purification procedure.

H. Paulsson et al., J. Phys. Chem. B 2003, 107, 13665-13670 relates to molten and solid trialkyl sulfonium iodides and their polyiodides as electrolytes in dye-synthesized nanocrystalline solar cells. The trialkyl sulfonium iodides, (R₂R'S)I, were synthesized from dialkyl sulfides (Me₂S, Et₂S, Pr₂S, Bu₂S, DodMeS) and alkyl iodides (MeI, EtI, 1-PrI, 1-BuI, 1-PeI, 1-HxI, 1-DodI) involving a nucleophilic attack by the sulfide on the alkyl iodide. The alkyl iodide was dissolved in acetone under light protection because of light-sensitivity of reactants. An equimolar amount of alkyl iodide was added and the reaction atmosphere was protected from oxygen and water by a continuous flow of dry nitrogen gas. The reaction mixture was left for several days at ambient conditions. The syntheses of the (R₂R'S)I salts were performed at room temperature. Higher reaction temperatures were also tested, resulting in decreased yields of the products.

WO 2016/073493 A2 relates to antifibrinylitic compounds, pharmaceutical and veterinary compositions thereof, and processes for their preparation. Trans-(4-dimethylthiomethyl) cyclohexane-1-carboxylic acid methyl ester sulfonium iodide is synthesized by dissolving trans-(4-chloromethyl) cyclohexane-1-carboxylic acid methyl ester in trifluoroacetic acid and treating the stirred solution overnight with an excess of potassium iodide. The TFA solution is filtered from insoluble salts, and a two-fold molar excess of dimethyl sulfide is added thereto, with warming to reflux. Following two hours of reaction, the solution is treated with cold diethyl ether, whereupon the precipitated product sulfonium iodide is obtained as an off-white powder.

As iodide salts and methosulfate salts of 1-octadecyldimethylsulfonium are not favored for infield agrochemical use, these syntheses would require an additional step of exchanging iodide or methosulfate against another more favorable anion such as chloride.

There is a demand for synthetic concepts that provide dimethyl-C₁₇₋₃₂-alkyl sulfonium salts, preferably dimethyl-C₁₇₋₃₂-alkyl sulfonium halides, more preferably dimethyl-C₁₇₋₃₂-alkyl sulfonium chlorides, and that have advantages over the synthetic concepts of the prior art, particularly with respect to the costs of the starting materials, yields, reaction times and required work-up.

It is an object of the invention to provide a process for the synthesis of dimethyl-C₁₇₋₃₂-alkyl sulfonium salts, preferably dimethyl-C₁₇₋₃₂-alkyl sulfonium halides, more preferably dimethyl-C₁₇₋₂-alkyl sulfonium chlorides, having advantages over the prior art.

This object has been achieved by the subject-matter of the patent claims.

It has been surprisingly found that dimethyl-C₁₇₋₃₂-alkyl sulfonium salts, preferably dimethyl-C₁₇₋₃₂-alkyl sulfonium halides, more preferably dimethyl-C₁₇₋₃₂-alkyl sulfonium chlorides, can be obtained at high yields in short reaction times employing inexpensive starting materials thereby making dimethyl-C₁₇₋₃₂-alkyl sulfonium salts, preferably dimethyl-C₁₇₋₃₂-alkyl sulfonium halides, more preferably dimethyl-C₁₇₋₃₂-alkyl sulfonium chlorides, available on industrial scale at reasonable costs.

When converting C₁₇₋₃₂-alkyl-alcohols into the corresponding C₁₇₋₃₂-alkyl-halides and using said C₁₇₋₃₂-alkyl-halides as starting materials in a subsequent reaction with dimethyl sulfide, the chemical nature of the halide has a significant influence on the conversion. The quality as a leaving group in nucleophilic attacks increases in the order —F, —Cl, —Br and —I. This is the reason why alkyl iodides are conventionally used as preferred alkylating agents; the activation energy for the corresponding reactions with alkyl chlorides (and in many instances also with alkyl bromides) would be too high in order to provide satisfactory yields within reasonable reaction times. Alkyl iodides, however, are disadvantageous not only because of light sensitivity and for environmental reasons, but especially also because of high costs. It is thus a significant advantage that according to the invention the C₁₇₋₃₂-alkyl-alcohols can be converted into the corresponding C₁₇₋₃₂-alkyl-chlorides and subsequently be reacted with dimethyl sulfide at excellent yields within reasonable reaction times in spite of the poorer leaving group quality of —Cl compared to —I. The formation of the C₁₇₋₃₂-alkyl-chlorides is inexpensive.

Further, it has been surprisingly found that dimethyl-C₁₇₋₃₂-alkyl sulfonium salts, preferably dimethyl-C₁₇₋₃₂-alkyl sulfonium halides, more preferably dimethyl-C₁₇₋₃₂-alkyl sulfonium chlorides, can be obtained from starting materials derived of natural sources, thereby providing all the advantages of green chemistry. Furthermore, the use of dimethyl sulfide avoids classical methylating agents such as iodomethane, dimethyl sulfate, or diazomethane, which are typically toxic and carcinogenic.

In particular, it has been surprisingly found that dimethyl-C₁₇₋₃₂-alkyl sulfonium salts, preferably dimethyl-C₁₇₋₃₂-alkyl sulfonium halides, more preferably dimethyl-C₁₇₋₃₂-alkyl sulfonium chlorides, can be obtained by reacting dimethyl sulfide with C₁₇₋₃₂-alkyl halides, preferably C₁₇₋₃₂-alkyl chlorides, in the presence of acid, preferably trifluoroacetic acid. The thus formed dimethyl-C₁₇₋₃₂-alkyl sulfonium acid salts, preferably trifluoroacetates, can subsequently be converted into the desired dimethyl-C₁₇₋₃₂-alkyl sulfonium salts, preferably dimethyl-C₁₇₋₃₂-alkyl sulfonium halides, more preferably dimethyl-C₁₇₋₃₂-alkyl sulfonium chlorides. It has been surprisingly found that when proceeding along these lines, dimethyl-C₁₇₋₃₂-alkyl sulfonium salts, preferably dimethyl-C₁₇₋₃₂-alkyl sulfonium halides, more preferably dimethyl-C₁₇₋₃₂-alkyl sulfonium chlorides, can be obtained in high yields and short reaction times.

### BRIEF DESCRIPTION OF THE DRAWINGS

The invention will now be described in greater detail with reference to the drawings, wherein:

FIG. 1 and FIG. 2 are ¹HNMR and ¹³CNMR spectra for the material obtained in Example A-3;

FIG. 3 and FIG. 4 are ¹HNMR and ¹⁹FNMR spectra for a material obtained in Example B-6;

FIG. 5 and FIG. 6 are ¹HNMR and ¹⁹FNMR spectra for another material obtained in Example B-6;

FIG. 7 is the ¹HNMR spectrum for a material obtained in Example C-4; and

FIG. 8, FIG. 9, and FIGS. 10A-H are ¹HNMR, ¹³CNMR and LCMS spectra for another material obtained in Example C-4.

The nucleophilic attack involving C₁₇₋₃₂-alkyl-chlorides as starting materials has an activation energy which is expected to be significantly higher than that involving C₁₇₋₃₂-alkyl-iodides instead. Thus, one would typically expect that the reaction of C₁₇₋₃₂-alkyl-chlorides with dimethyl sulfide requires extended reaction times under harsh reaction conditions providing poor yields. Without wishing to be bound to any scientific theory, it appears that the presence of an acid, preferably trifluoroacetic acid, lowers the activation energy such that high yields can be obtained within reasonable reaction times.

Further, it has been surprisingly found that an excess of dimethyl sulfide relative to the C₁₇₋₃₂-alkyl halide provides higher yields at comparatively short reaction times. Moreover, the excess of dimethyl sulfide can be easily recovered since dimethyl sulfide has a low boiling point compared to the other reactants and/or solvents. This is particularly advantageous because the two reactants simply react with one another in an equimolar ratio without providing byproducts such as methyl sulfuric acid that would otherwise be obtained as a byproduct when using dimethyl sulfate as a methylating agent. Therefore, the process according to the invention does not require laborious purification procedures in order to separate such byproducts from the desired products.

Still further, it has been surprisingly found that the use of an acid, preferably of trifluoroacetic acid, as a solvent significantly improves the yield compared to a stoichiometric use of acid, preferably trifluoroacetic acid, based on the C₁₇₋₃₂-alkyl halide. Moreover, the excess of acid, preferably trifluoroacetic acid, can easily be recovered since its boiling point is different from that of the other reactants. Thus, the comparatively high costs of trifluoroacetic acid do not negatively influence the overall costs of the process; the trifluoroacetic acid can be recycled and is not consumed.

Furthermore, it has been surprisingly found that isolation of the intermediately formed dimethyl-C₁₇₋₃₂-alkyl sulfonium acid salt, preferably trifluoroacetate, prior to conversion into the desired dimethyl-C₁₇₋₃₂-alkyl sulfonium salt, preferably dimethyl-C₁₇₋₃₂-alkyl sulfonium halide, more preferably dimethyl-C₁₇₋₃₂-alkyl sulfonium chloride, significantly improves the purity of the dimethyl-C₁₇₋₃₂-alkyl sulfonium salt, preferably the dimethyl-C₁₇₋₃₂-alkyl sulfonium halide, more preferably dimethyl-C₁₇₋₃₂-alkyl sulfonium chloride, thus obtained.

Moreover, it has been surprisingly found that the intermediately formed dimethyl-C₁₇₋₃₂-alkyl sulfonium acid salt, preferably trifluoroacetate, into the desired end product, i.e. dimethyl-C₁₇₋₃₂-alkyl sulfonium salt, preferably a dimethyl-C₁₇₋₃₂-alkyl sulfonium halide, more preferably a dimethyl-C₁₇₋₃₂-alkyl sulfonium chloride, still more preferably dimethyl octadecyl sulfonium chloride, can be easily performed in high yield and purity. The intermediately formed dimethyl-C₁₇₋₃₂-alkyl sulfonium acid salt, preferably trifluoroacetate, makes thus available a broad variety of different dimethyl-C₁₇₋₃₂-alkyl sulfonium salts simply by exchanging the anion, preferably trifluoroacetate, against another desired anion.

In particularly preferred embodiments of the invention, the target molecule to be prepared by the process according to the invention is a dimethyl-C₁₇₋₃₂-alkyl sulfonium halide, more preferably a dimethyl-C₁₇₋₃₂-alkyl sulfonium chloride, still more preferably dimethyl octadecyl sulfonium chloride.

Unexpectedly, the trialkyl sulfonium chlorides, preferably octadecyl dimethyl sulfonium chloride according to the invention are superior over the respective bromides and iodides, the latter being discussed in WO 2020/201698 A1. While with respect to depolarization of mitochondria and ability to induce mitochondrial ROS formation no significant differences could be observed for the chlorides, bromides and iodides, the chlorides according to the invention provide significant benefit with respect to inducing apoptotic cell death compared to the respective bromides and iodides.

Furthermore, it has been surprisingly found that trialkyl sulfonium salts having a minimum alkyl chain length C18 (octadecyl, stearyl) at residue R3 are capable of suppressing mitochondrial activity, whereas comparative trialkyl sulfonium salts having shorter alkyl chain length at residue R3 show no corresponding effect, irrespective of the counter anion. There is experimental indication that certain salts of trialkyl sulfonium, particularly the trialkyl sulfonium chlorides, are more stable than others, especially under UV-light.

A first aspect of the invention relates to a process for the preparation of a dimethyl-C₁₇₋₃₂-alkyl sulfonium salt, preferably a dimethyl-C₁₇₋₃₂-alkyl sulfonium halide, more preferably a dimethyl-C₁₇₋₃₂-alkyl sulfonium chloride, still more preferably dimethyl octadecyl sulfonium chloride;
preferably wherein the dimethyl-C₁₇₋₃₂-alkyl sulfonium salt, preferably the dimethyl-C₁₇₋₃₂-alkyl sulfonium halide has general formula (VI)

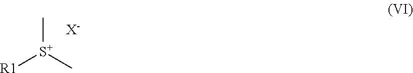

wherein R1 represents —(CH₂)ₙ—CH₃, wherein n is an integer within the range of from 16 to 31, preferably 17; and X is selected from the group consisting of —F, —Cl, —Br, and —I, preferably —Cl;
   the method comprising the steps of:
(a) optionally, providing a mixture of a C₁₇₋₃₂-alkyl alcohol and a halide donor;
   preferably wherein the C₁₇₋₃₂-alkyl alcohol has general formula (I) R1—OH, wherein R1 represents —(CH₂)ₙ—CH₃, wherein n is an integer within the range of from 16 to 31, preferably 17; and
   preferably wherein the acid halide has general formula (II)

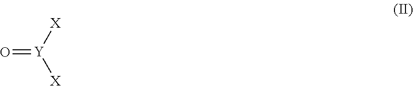

wherein Y is selected from C and S, preferably S; and X is selected from the group consisting of —F, —Cl, —Br, and —I, preferably —Cl;
(b) optionally, reacting the C₁₇₋₃₂-alkyl alcohol with the halide donor thereby providing a C₁₇₋₃₂-alkyl halide;
(c) providing a mixture of the C₁₇₋₃₂-alkyl halide, dimethyl sulfide, and preferably an acid;
   preferably wherein the C₁₇₋₃₂-alkyl halide has general formula (III) R1-X, wherein R1 represents —(CH₂)ₙ—CH₃, wherein n is an integer within the range of from 16 to 31, preferably 17; and X is selected from the group consisting of —F, —Cl, —Br, and —I, preferably —Cl;
   preferably wherein the acid is a carboxylic acid having general formula (IV)

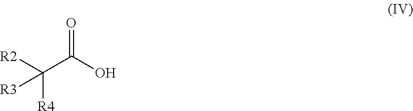

wherein R2, R3 and R4 independently of one another represent —H, —Cl, or —F, preferably —F; preferably with the proviso that at least one of R2, R3 and R4 does not represent —H; preferably wherein step (c) comprises the sub-steps of:
(c-1) providing a composition comprising the C₁₇₋₃₂-alkyl halide and a solvent; and
(c-2) adding the dimethyl sulfide to the composition provided in step (c-1);
(d) reacting the C₁₇₋₃₂-alkyl halide with the dimethyl sulfide;
   preferably wherein step (d) comprises the sub-steps of:
   (d-1) reacting the C₁₇₋₃₂-alkyl halide and dimethyl sulfide in the presence of the acid thereby providing a dimethyl-C₁₇₋₃₂-alkyl sulfonium acid salt;
      preferably wherein the dimethyl-C₁₇₋₃₂-alkyl sulfonium acid salt has general formula (V):

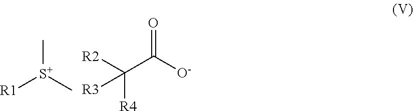

wherein R1 represents —(CH₂)ₙ—CH₃, wherein n is an integer within the range of from 16 to 31, preferably 17; and wherein R2, R3 and R4 independently of one another represent —H, —Cl, or —F; preferably with the proviso that at least one of R2, R3 and R4 does not represent —H; more preferably wherein R2, R3 and R4 represent —F;
(d-2) separating at least the majority of the dimethyl-C₁₇₋₃₂-alkyl sulfonium acid salt from the product composition obtained in sub-step (d-1);
(d-3) optionally, recycling the product composition from which at least the majority of the dimethyl-C₁₇₋₃₂-alkyl sulfonium acid salt has been separated in sub-step (d-2) to the mixture provided in step (c); and
(d-4) converting the dimethyl-C₁₇₋₃₂-alkyl sulfonium acid salt into the dimethyl-C₁₇₋₃₂-alkyl sulfonium salt, preferably the dimethyl-C₁₇₋₃₂-alkyl sulfonium halide.

In preferred embodiments, the process according to the invention comprises the steps of:
(c) providing a mixture of a C₁₇₋₃₂-alkyl halide and dimethyl sulfide; and
(d) reacting the C₁₇₋₃₂-alkyl halide with the dimethyl sulfide.

In preferred embodiments, the process according to the invention comprises the preceding steps of:
(a) providing a mixture of a C₁₇₋₃₂-alkyl alcohol and a halide donor; and
(b) reacting the C₁₇₋₃₂-alkyl alcohol with the halide donor thereby providing the C₁₇₋₃₂-alkyl halide.

In preferred embodiments, step (a) of the process according to the invention comprises the substeps of:
(a-1) dissolving the C₁₇₋₃₂-alkyl alcohol in a first organic solvent, preferably in dichloromethane;
(a-2) adding a second organic solvent to the mixture thus obtained in step (a-1), preferably N,N-dimethylformamide; and
(a-3) adding the halide donor to the mixture thus obtained in step (a-2).

In preferred embodiments, step (b) of the process according to the invention comprises the substeps of:
(b-1) heating the mixture provided in step (a), preferably to a temperature of about 50° C.;
(b-2) allowing the C₁₇₋₃₂-alkyl alcohol and the halide donor to react thereby providing the C₁₇₋₃₂-alkyl halide in a product mixture, preferably in a time of about 24 hours; and
(b-3) optionally, recovering at least the majority of the C₁₇₋₃₂-alkyl halide from the product mixture thus obtained in step (b-2).

Preferably, the recovering in sub-step (b-3) involves solvent extraction, filtration, washing, drying and/or recrystallization.

A preferred work-up procedure of the C₁₇₋₃₂-alkyl halide according to the invention includes
(i) concentrating the product mixture obtained in sub-step (b-3), preferably under reduced pressure, preferably at elevated temperature;
(ii) optionally, removing of volatile compounds from the residue thus obtained in step (i) under reduced pressure;
(iii) dissolving the residue thus obtained in step (i) or step (ii) in a mixture of two organic solvents having different polarities; preferably a mixture of hexane and methyl tert-butyl ether, preferably in a volume ratio of hexane to methyl tert-butyl ether of 9:1;
(iv) filtering the mixture thus obtained in step (iii) through a filter aid, preferably silica; and
(v) removing of volatile compounds from the filtrate thus obtained in step (iv), preferably under reduced pressure, to obtain the C₁₇₋₃₂-alkyl halide.

Preferably, the C₁₇₋₃₂-alkyl alcohol has general formula (I):

R1—OH (I)

wherein R1 represents —(CH₂)ₙ—CH₃, wherein n is an integer within the range of from 16 to 31.

Preferably, R1 in general formula (I) is —(CH₂)₁₇—CH₃.

Preferably, the halide donor is an acid halide.

Preferably, the acid halide has general formula (II)

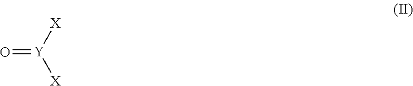

wherein
Y is selected from C and S; and
X is selected from the group consisting of —F, —Cl, —Br, and —I.

Preferably, Y in general formula (II) is S and X in general formula (II) is —Cl.

Preferably, the halide donor according to the invention is thionyl chloride.

Preferably, the C₁₇₋₃₂-alkyl halide has general formula (III)

R1—X (III)

wherein
R1 represents —(CH₂)ₙ—CH₃, wherein n is an integer within the range of from 16 to 31; and
X is selected from the group consisting of —F, —Cl, —Br, and —I.

Preferably, R1 in general formula (III) is —(CH₂)₁₇—CH₃.

Preferably, X in general formula (III) is —Cl.

Preferably, the C₁₇₋₃₂-alkyl halide according to the invention is 1-chlorooctadecane.

In preferred embodiments, the mixture provided in step (c) additionally comprises an acid differing from HF, HCl, HBr and HI.

Preferably, the acid of the mixture provided in step (c) is a carboxylic acid.

Preferably, the carboxylic acid has general formula (IV)

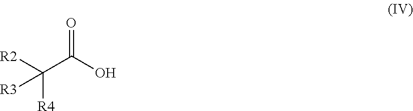

wherein R2, R3 and R4 independently of one another represent —H, —Cl, or —F; preferably with the proviso that at least one of R2, R3 and R4 does not represent —H.

Preferably, R2, R3 and R4 in general formula (IV) are —F.

Preferably, the acid according to the invention is trifluoroacetic acid.

In preferred embodiments, the acid and the C₁₇₋₃₂-alkyl halide are provided in step (c) in a molar ratio of the acid to the C₁₇₋₃₂-alkyl halide of
at least 3.5, preferably at least 5.0, more preferably at least 7.5, still more preferably at least 10, yet more preferably at least 13, even more preferably at least 15, least preferably at least 17, and in particular at least 19; and/or
within the range of 4.0±0.5, or 5.0±1.0, or 5.0±0.5, or 7.5±2.5, or 7.5±1.0, or 7.5±0.5, or 10±5.0, or 10±2.5, or 10±1.0, or 10±0.5, or 13±7.5, or 13±5.0, or 13±2.5, or 13±1.0, or 13±0.5, or 19±10, or 19±7.5, or 19±5.0, 19±2.5, or 19±1.0, or 19±0.5.

In preferred embodiments, step (c) of the process according to the invention comprises the substep of:
(c-1) providing a composition comprising the C₁₇₋₃₂-alkyl halide and a solvent.

Preferably, the solvent of the composition provided in sub-step (c-1) is the acid as described above.

In preferred embodiments, the C₁₇₋₃₂-alkyl halide is provided in the composition in sub-step (c-1) in a concentration of
at most 3.2 mol/L, preferably at most 3.0 mol/L, more preferably at most 2.7 mol/L, still more preferably at most 2.4 mol/L, yet more preferably at most 2.1 mol/L, even more preferably at most 1.8 mol/L, most preferably at most 1.5 mol/L, and in particular at most 0.99 mol/L; and/or
within the range of 1.0±0.5 mol/L, or 1.25±0.75 mol/L, or 1.25±0.5 mol/L, or 1.5±1.0 mol/L, or 1.5±0.75 mol/L, or 1.5±0.5 mol/L, or 1.75±1.25 mol/L, or 1.75±1.0 mol/L, or 1.75±0.75 mol/L, or 1.75±0.5 mol/L, or 2.0±1.30 mol/L, or 2.0±1.25 mol/L, or 2.0±1.0 mol/L, or 2.0±0.75 mol/L, or 2.0±0.5 mol/L.

Preferably, step (c) of the process according to the invention comprises the sub-step of:
(c-2) adding the dimethyl sulfide to the composition provided in sub-step (c-1).

In preferred embodiments, the dimethyl sulfide is provided in the composition in sub-step (c-2) in a concentration of
at most 1.68 mol/L, preferably at most 1.30 mol/L, more preferably at most 1.05 mol/L, still more preferably at most 0.90 mol/L, yet more preferably at most 0.75 mol/L, even more preferably at most 0.60 mol/L, most preferably at most 0.45 mol/L, and in particular at most 0.30 mol/L; and/or
within the range of 0.5±0.25 mol/L, or 0.75±0.5 mol/L, or 0.75±0.25 mol/L, or 1.0±0.65 mol/L, or 1.0±0.5 mol/L, or 1.0±0.25 mol/L.

In preferred embodiments, the dimethyl sulfide and the C₁₇₋₃₂-alkyl halide are provided in step (c) in a molar ratio of dimethyl sulfide to C₁₇₋₃₂-alkyl halide of
at least 0.52, preferably at least 0.67, more preferably at least 0.82, still more preferably at least 0.97, yet more preferably at least 1.12, even more preferably at least 1.27, least preferably at least 1.42, and in particular at least 1.5; and/or
within the range of 0.75±0.23, or 1.0±0.48, or 1.0±0.23, or 1.25±0.73, or 1.25±0.48, or 1.25±0.23, or 1.5±0.98, 1.5±0.73, or 1.5±0.48, or 1.5±0.23.

In preferred embodiments, step (d) of the process according to the invention comprises the substep of:
(d-1) reacting the C₁₇₋₃₂-alkyl halide and dimethyl sulfide in the presence of the acid thereby providing a dimethyl-C₁₇₋₃₂-alkyl sulfonium acid salt.

Preferably, the dimethyl-C₁₇₋₃₂-alkyl sulfonium acid salt has general formula (V):

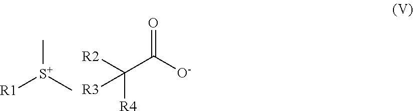

wherein
R1 represents —(CH₂)ₙ—CH₃, wherein n is an integer within the range of from 16 to 31; and
wherein R2, R3 and R4 independently of one another represent —H, —Cl, or —F; preferably with the proviso that at least one of R2, R3 and R4 does not represent —H.

Preferably, R1 in general formula (V) is —(CH₂)₁₇—CH₃, and R2, R3 and R4 in general formula (V) are —F.

Preferably, the dimethyl-C₁₇₋₃₂-alkyl sulfonium acid salt according to the invention is dimethyloctadecyl sulfonium trifluoroacetate.

In preferred embodiments, the dimethyl-C₁₇₋₃₂-alkyl sulfonium acid salt according to the invention is not a dimethyl-C₁₇₋₃₂-alkyl sulfonium halide.

In other preferred embodiments, the dimethyl-C₁₇₋₃₂-alkyl sulfonium acid salt according to the invention is a halide, which then in sub-step (d-4) is converted to another dimethyl-C₁₇₋₃₂-alkyl sulfonium salt, preferably another dimethyl-C₁₇₋₃₂-alkyl sulfonium halide.

Preferably, sub-step (d-1) is performed under elevated temperature.

In preferred embodiments, sub-step (d-1) is performed at a temperature of
at least 71° C., preferably at least 80° C., more preferably at least 85° C., still more preferably at least 91° C., yet more preferably at least 95° C., even more preferably at least 100° C., most preferably at least 105° C., and in particular at least 110° C.; and/or
within the range of 81±10° C., or 86±15° C., or 86±10° C., or 91±20° C., or 91±15° C., or 91±10° C., or 96±25° C., or 96±20° C., or 96±15° C., or 96±10° C., or 101±30° C., or 101±25° C., or 101±20° C., or 101±15° C., or 101±10° C., or 106±35° C., or 106±30° C., or 106±25° C., or 106±20° C., or 106±15° C., or 106±10° C., or 111±40° C., or 111±35° C. or 111±30° C., or 111±25° C., or 111±20° C., or 111±15° C., or 111±10° C.

Preferably, sub-step (d-1) is performed under elevated pressure.

Preferably, sub-step (d-1) is performed in an autoclave at a temperature within the range of from 100 to 120° C.; preferably at about 110±2° C.

In preferred embodiments, sub-step (d-1) is performed in a time of
at most 168 h, preferably at most 124 h, more preferably at most 120 h, still more preferably at most 96 h, yet more preferably at most 72 h, even more preferably at most 48 h, most preferably at most 36 h, and in particular at most 24 h; and/or
within the range of 12±6 h, or 18±12 h, or 18±6 h, or 24±18 h, or 24±12 h, or 24±6 h, or 30±24 h, or 30±18 h, or 30±12 h, or 30±6 h, or 36±30 h, or 36±24 h, or 36±18 h, or 36±12 h, or 36±6 h, 42±36 h, or 42±30 h, or 42±24 h, or 42±18 h, or 42±12 h, or 42±6 h.

Preferably, in sub-step (d-1) a product composition is obtained comprising the dimethyl-C₁₇₋₃₂-alkyl sulfonium acid salt and optionally remaining C₁₇₋₃₂-alkyl halide, acid and/or dimethyl sulfide.

Preferably, step (d) comprises the sub-step of:
(d-2) separating at least the majority of the dimethyl-C₁₇₋₃₂-alkyl sulfonium acid salt from the product composition obtained in sub-step (d-1).

Preferably, sub-step (d-2) involves filtration, solvent extraction, ion-exchange chromatography, washing, drying, and/or recrystallization.

A preferred work-up procedure of the dimethyl-C₁₇₋₃₂-alkyl sulfonium acid salt according to the invention includes
(i) optionally, filtering the product composition and washing the filtrate thus obtained in step (i) with an organic solvent, preferably with methyl tert-butyl ether;
(ii) concentrating the product composition or the filtrate thus obtained in step (i), preferably under reduced pressure, preferably at elevated temperature;
(iii) optionally, removing of volatile compounds from the residue thus obtained in step (ii) under reduced pressure;
(iv) dissolving the residue thus obtained in step (ii) or step (iii) in a mixture of two organic solvents, preferably a mixture of diethyl ether and pentane, preferably in a volume ratio of diethyl ether to pentane of 1:1; preferably at elevated temperature, more preferably at 40° C.;
(v) optionally, filtering the mixture thus obtained in step (iv);
(vi) cooling the mixture thus obtained in step (iv) or the filtrate thus obtained in step (v), preferably to a temperature of about −50° C., thereby precipitating at least the majority of the dimethyl-C₁₇₋₃₂-alkyl sulfonium acid salt;
(vii) filtering the precipitate thus obtained in step (vi) and washing the feed thus obtained in step (vii) first with a mixture of two organic solvents, preferably a mixture of diethyl ether and pentane, preferably in a volume ratio of diethyl ether to pentane of 1:1, and secondly with an organic solvent, preferably with pentane; and
(viii) optionally, drying the filtered material thus obtained in step (vii) to obtain the dimethyl-C₁₇₋₃₂-alkyl sulfonium acid salt.

Preferably, step (d) comprises the sub step of:
(d-3) recycling the product composition from which at least the majority of the dimethyl-C₁₇₋₃₂-alkyl sulfonium acid salt has been separated in sub-step (d-2) to the mixture provided in step (c).

Preferably, step (d) comprises the sub-step of:
(d-4) converting the dimethyl-C₁₇₋₃₂-alkyl sulfonium acid salt separated in sub-step (d-2) into the dimethyl-C₁₇₋₃₂-alkyl sulfonium salt, preferably the dimethyl-C₁₇₋₃₂-alkyl sulfonium halide.

In preferred embodiments, the conversion in sub-step (d-4) involves contacting the dimethyl-C₁₇₋₃₂-alkyl sulfonium acid salt with a halide donor according to the invention as defined above.

In other preferred embodiments, the conversion in sub-step (d-4) involves contacting the dimethyl-C₁₇₋₃₂-alkyl sulfonium acid salt with a further acid.

In these preferred embodiments, the acid of the mixture provided in step (c) is preferably different from the further acid of sub-step (d-4).

Preferably, the further acid is
an inorganic acid, preferably a mineral acid, more preferably selected from HF, HCl, HBr and HI; or
an organic acid, preferably a carboxylic acid, more preferably a C₁₋₃₂-carboxylic acid, and still more preferably oxalic acid or stearic acid.

Preferably, the dimethyl-C₁₇₋₃₂-alkyl sulfonium salt, preferably the dimethyl-C₁₇₋₃₂-alkyl sulfonium halide has general formula (VI)

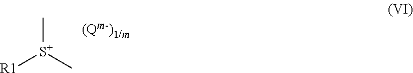

wherein
R1 represents —(CH₂)ₙ—CH₃, wherein n is an integer within the range of from 16 to 31;
Q is X or the conjugate base of the further acid, wherein X is selected from the group consisting of —F, —Cl, —Br, and —I; and
m is an integer of 1, 2, or 3.

Preferably, R1 in general formula (VI) is —(CH₂)₁₇—CH₃.

Preferably, Q in general formula (VI) is X.

Preferably, when Q in general formula (VI) is X, X is —Cl.

Preferably, the integer m in general formula (VI) is 1.

Preferably, the dimethyl-C₁₇₋₃₂-alkyl sulfonium salt, preferably the dimethyl-C₁₇₋₃₂-alkyl sulfonium halide according to the invention is dimethyl-octadecyl sulfonium chloride.

In preferred embodiments, Q^{m-} is a monovalent anion (m=1).

In other preferred embodiments, Q^{m-} is a divalent anion (m=2).

In further preferred embodiments, Q^{m-} is a trivalent anion (m=3).

In preferred embodiments, Q^{m-} is the conjugate base of an inorganic acid, i.e. said further acid is preferably an inorganic acid.

In other preferred embodiments, Q^{m-} is the conjugate base of an organic acid, i.e. said further acid is preferably an organic acid. In preferred embodiments, the organic acid is aliphatic. In other preferred embodiments, the organic acid is aromatic.

In preferred embodiments, Q^{m-} is the conjugate base of an acid carrying at least one acidic functional group selected from —CO₂H, —SO₃H, and —OSO₃H, i.e. said further acid is preferably an acid carrying at least one acidic functional group selected from —CO₂H, —SO₃H, and —OSO₃H.

In preferred embodiments, Q^{m-} is the conjugate base of water (i.e. hydroxide, HO⁻), i.e. said further acid is preferably water.

In preferred embodiments, Q^{m-} is the conjugate base of a mineral acid, i.e. said further acid is preferably a mineral acid; preferably selected from the group consisting of HBF₄, HBO₂, HBO₃, H₃BO₃, H₂CO₃, H₄SiO₄, HNO₃, H₃PO₃, H₃PO₄, H₂S, H₂SO₃, HSO₃F, H₂SO₄, HF, HCl, HBr, HI, HClO₃, and HClO₄. Thus, Q^{m-} is preferably selected from tetrafluoroborate, metaborate, perborate, borate, hydrogen carbonate, carbonate, silicate, nitrate, hydrogen phosphite, phosphite, dihydrogen phosphate, hydrogen phosphate, phosphate (orthophosphate), hydrogen sulfide, sulfide, hydrogen sulfite, sulfite, fluorosulfonate, hydrogen sulfate, sulfate, fluoride, chloride, bromide, iodide, chlorate and perchlorate.

In preferred embodiments, Q^{m-} is the conjugate base of a monocarboxylic acid, i.e. said further acid is preferably a monocarboxylic acid.

In preferred embodiments, Q^{m-} is the conjugate base of a saturated aliphatic monocarboxylic acid, i.e. said further acid is preferably a saturated aliphatic monocarboxylic acid; preferably selected from the group consisting of formic acid, acetic acid, propionic acid, butyric acid, valeric acid, caproic acid, enanthic acid, pelargonic acid, capric acid, undecylic acid, lauric acid, tridecylic acid, myristic acid, pentadecylic acid, palmitic acid, margaric acid, stearic acid, nonadecylic acid, and arachidic acid. Thus, Q^{m-} is preferably selected from formate, acetate, propionate, butyrate, valerate, caprylate, enanthate, pelargonate, caprate, undecylate, laurate, tridecylate, myristate, pentadecylate, palmitate, margarate, stearate, nonadecylate, arachidate.

In preferred embodiments, Q^{m-} is the conjugate base of an unsaturated aliphatic monocarboxylic acid, i.e. said further acid is preferably an unsaturated aliphatic monocarboxylic acid; preferably selected from the group consisting of acrylic acid, methacrylic acid, crotonic acid, and oleic acid. Thus, Q^{m-} is preferably selected from acrylate, methacrylate, crotonate and oleate.

In preferred embodiments, Q^{m-} is the conjugate base of an aromatic monocarboxylic acid, i.e. said further acid is preferably an aromatic monocarboxylic acid; preferably benzoic acid. Thus, (Q^{m-})_{1/m} is preferably benzoate. The aromatic monocarboxylic acid, preferably benzoic acid, may optionally be substituted with 1, 2 or 3 substituents independently of one another selected from —F, —Cl, —OH, —OCH₃, —CH₃, —CN, and —NO₂.

In other preferred embodiments, Q^{m-} is the conjugate base of a dicarboxylic acid, i.e. said further acid is preferably a dicarboxylic acid.

In preferred embodiments, Q^{m-} is the conjugate base of a saturated aliphatic dicarboxylic acid, i.e. said further acid is preferably a saturated aliphatic dicarboxylic acid; preferably selected from the group consisting of oxalic acid, malonic acid, succinic acid, glutaric acid, adipic acid, pimelic acid, suberic acid, azelaic acid, and sebacic acid. Thus, Q^{m-} is preferably selected from hydrogen oxalate, oxalate, hydrogen malonate, malonate, hydrogen succinate, succinate, hydrogen glutarate, glutarate, hydrogen adipate, adipate, hydrogen pimelate, pimelate, hydrogen suberate, suberate, hydrogen azelate, azelate, hydrogen sebacate and sebacate.

In preferred embodiments, Q^{m-} is the conjugate base of an unsaturated aliphatic dicarboxylic acid, i.e. said further acid is preferably an unsaturated aliphatic dicarboxylic acid; preferably selected from the group consisting of maleic acid, fumaric acid, glutaconic acid, muconic acid, citraconic acid, mesaconic acid, and itaconic acid. Thus, Q^{m-} is preferably selected from hydrogen maleate, maleate, hydrogen fumarate, fumarate, hydrogen glutaconate, glutaconate, hydrogen muconate, muconate, hydrogen citraconate, citraconate, hydrogen mesaconate, mesaconate, hydrogen itaconate and itaconate.

In preferred embodiments, Q^{m-} is the conjugate base of an aromatic dicarboxylic acid, i.e. said further acid is preferably an aromatic dicarboxylic acid; preferably selected from the group consisting of phthalic acid, isophthalic acid, and terephthalic acid. Thus, Q^{m-} is preferably selected from hydrogen phthalate, phthalate, hydrogen isophthalate, isophthalate, hydrogen terephthalate and terephthalate.

In preferred embodiments, Q^{m-} is the conjugate base of a hydroxycarboxylic acid, i.e. said further acid is preferably a hydroxycarboxylic acid; preferably selected from the group consisting of glycolic acid, lactic acid, malic acid, tartaric acid, citric acid, and mandelic acid. Thus, Q^{m-} is preferably selected from glycolate, lactate, malate, hydrogen tartrate, tartrate, dihydrogen citrate, hydrogen citrate, citrate and mandelate.

In preferred embodiments, Q^{m-} is the conjugate base of a keto carboxylic acid, i.e. said further acid is preferably a keto carboxylic acid; preferably selected from the group consisting of pyruvic acid, acetoacetic acid, and levulinic acid. Thus, Q^{m-} is preferably selected from pyruvate, acetoacetate and levulate.

In preferred embodiments, Q^{m-} is the conjugate base of a halogenated carboxylic acid, i.e. said further acid is preferably a halogenated carboxylic acid; preferably selected from the group consisting of fluoroacetic acid, difluoroacetic acid, trifluoroacetic acid, chloroacetic acid, dichloroacetic acid, trichloroacetic acid. Thus, Q^{m-} is preferably selected from fluoroacetate, difluoroacetate, trifluoroacetate, chloroacetate, dichloroacetate and trichloroacetate.

In preferred embodiments, Q^{m-} is the conjugate base of an amino acid, i.e. said further acid is preferably an amino acid; preferably selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. Thus, Q^{m-} is preferably selected from alaninate, argininate, asparaginate, aspartate, cysteinate, glutaminate, glutamate, glycinate, histidinate, isoleucinate, leucinate, lysinate, methioninate, phenylalaninate, prolinate, serinate, threoninate, tryptophanate, tyrosinate, and valinate.

In preferred embodiments, Q^{m-} is the conjugate base of an alkyl hydrogen sulfate, i.e. said further acid is preferably an alkyl hydrogen sulfate; preferably methyl hydrogen sulfate. Thus, Q^{m-} is preferably methyl sulfate.

In preferred embodiments, Q^{m-} is the conjugate base of an alkyl sulfonic acid, i.e. said further acid is preferably an alkyl sulfonic acid; preferably selected from methyl sulfonic acid, trifluoromethyl sulfonic acid, and ethyl sulfonic acid. Thus, Q^{m-} is preferably selected from methyl sulfonate, trifluoromethyl sulfonate and ethyl sulfonate.

In preferred embodiments, Q^{m-} is the conjugate base of an aryl sulfonic acid, i.e. said further acid is preferably an aryl sulfonic acid; preferably selected from benzene sulfonic acid, and p-toluene sulfonic acid. Thus, Q^{m-} is preferably selected from benzene sulfonate and p-toluene sulfonate.

A particularly preferred dimethyl-C₁₇₋₃₂-alkyl sulfonium salt according to the invention is noctadecyl dimethyl sulfonium
a. hydroxide;
b. tetrafluoroborate, metaborate, perborate, borate, hydrogen carbonate, carbonate, silicate, nitrate, hydrogen phosphite, phosphite, dihydrogen phosphate, hydrogen phosphate, phosphate (orthophosphate), hydrogen sulfide, sulfide, hydrogen sulfite, sulfite, fluorosulfonate, hydrogen sulfate, sulfate, fluoride, chloride, bromide, iodide, chlorate, perchlorate;
c. formate, acetate, propionate, butyrate, valerate, caprylate, enanthate, pelargonate, caprate, undecylate, laurate, tridecylate, myristate, pentadecylate, palmitate, margarate, stearate, nonadecylate, arachidate;
d. acrylate, methacrylate, crotonate, oleate;
e. benzoate;
f. hydrogen oxalate, oxalate, hydrogen malonate, malonate, hydrogen succinate, succinate, hydrogen glutarate, glutarate, hydrogen adipate, adipate, hydrogen pimelate, pimelate, hydrogen suberate, suberate, hydrogen azelate, azelate, hydrogen sebacate, sebacate;
g. hydrogen maleate, maleate, hydrogen fumarate, fumarate, hydrogen glutaconate, glutaconate, hydrogen muconate, muconate, hydrogen citraconate, citraconate, hydrogen mesaconate, mesaconate, hydrogen itaconate, itaconate;
h. hydrogen phthalate, phthalate, hydrogen isophthalate, isophthalate, hydrogen terephthalate, terephthalate;
i. glycolate, lactate, malate, hydrogen tartrate, tartrate, dihydrogen citrate, hydrogen citrate, citrate, mandelate;
j. pyruvate, acetoacetate, levulate;
k. fluoroacetate, difluoroacetate, trifluoroacetate, chloroacetate, dichloroacetate, trichloroacetate;
l. alaninate, argininate, asparaginate, aspartate, cysteinate, glutaminate, glutamate, glycinate, histidinate, isoleucinate, leucinate, lysinate, methioninate, phenylalaninate, prolinate, serinate, threoninate, tryptophanate, tyrosinate, valinate;
m. methyl sulfate;
n. methyl sulfonate, trifluoromethyl sulfonate, ethyl sulfonate;
o. benzene sulfonate or p-toluene sulfonate.

In preferred embodiments, sub-step (d-4) of the process according to the invention includes
(i) dissolving the dimethyl-C₁₇₋₃₂-alkyl sulfonium acid salt in an acid and/or a solvent, preferably in a mixture of an acid and a solvent, more preferably in a mixture of HCl and methanol; (ii) optionally, cooling the mixture thus obtained in step (i), preferably to a temperature of about −10° C.;
(iii) adding the halide donor as defined above to the mixture thus obtained in step (i) or step (ii); (iv) optionally, heating the mixture thus obtained in step (iii), preferably to a temperature of about 40° C.;
(v) allowing the dimethyl-C₁₇₋₃₂-alkyl sulfonium acid salt and the halide donor to form the dimethyl-C₁₇₋₃₂-alkyl sulfonium salt, preferably the dimethyl-C₁₇₋₃₂-alkyl sulfonium halide, preferably for a time of about 15 h;
(vi) removing of volatile compounds from the mixture thus obtained in step (v) under reduced pressure;
(vii) optionally, recrystallizing the residue thus obtained in step (vi) from an organic solvent, preferably acetone; and
(viii) optionally, drying the residue thus obtained in step (vi) or step (vii) to obtain the dimethyl-C₁₇₋₃₂-alkyl sulfonium salt, preferably the dimethyl-C₁₇₋₃₂-alkyl sulfonium halide.

In other preferred embodiments, sub-step (d-4) of the process according to the invention includes
(i) dissolving the dimethyl-C₁₇₋₃₂-alkyl sulfonium acid salt in the further acid and/or a solvent, preferably in a mixture of the further acid and a solvent, more preferably in a mixture of HBr and methanol;
(ii) optionally, heating the mixture thus obtained in step (i), preferably to a temperature of about 50° C.;
(iii) allowing the dimethyl-C₁₇₋₃₂-alkyl sulfonium acid salt and the further acid to form the dimethyl-C₁₇₋₃₂-alkyl sulfonium salt, preferably the dimethyl-C₁₇₋₃₂-alkyl sulfonium halide, preferably for a time of about 15 h;
(iv) concentrating the mixture thus obtained in step (iii), preferably under reduced pressure;
(v) optionally, filtering the residue thus obtained in step (iv) and washing the feed thus obtained in step (v) with an organic solvent, preferably diethyl ether;
(vi) optionally, recrystallizing the residue thus obtained in step (v) from an organic solvent, preferably acetone; and
(vii) optionally, drying the residue thus obtained in any of steps (iv) to (vi) to obtain the dimethyl-C₁₇₋₃₂-alkyl sulfonium salt, preferably the dimethyl-C₁₇₋₃₂-alkyl sulfonium halide.

In other preferred embodiments, sub-step (d-4) of the process according to the invention includes
(i) dissolving the dimethyl-C₁₇₋₃₂-alkyl sulfonium acid salt in the further acid and/or a solvent, preferably in a mixture of the further acid and a solvent, more preferably in a mixture of HI and methanol;
(ii) optionally, heating the mixture thus obtained in step (i), preferably to a temperature of about 50° C.;
(iii) allowing the dimethyl-C₁₇₋₃₂-alkyl sulfonium acid salt and the further acid to form the dimethyl-C₁₇₋₃₂-alkyl sulfonium salt, preferably the dimethyl-C₁₇₋₃₂-alkyl sulfonium halide, preferably for a time of about 15 h;
(iv) concentrating the mixture thus obtained in step (iii), preferably under reduced pressure;
(v) optionally, filtering residue thus obtained in step (iv) and washing the feed thus obtained in step (v) with an organic solvent, preferably diethyl ether;
(vi) optionally, recrystallizing residue thus obtained in step (v) from an organic solvent, preferably acetone; and
(vii) optionally, drying the residue thus obtained in any of steps (iv) to (vi) to obtain the dimethyl-C₁₇₋₃₂-alkyl sulfonium salt, preferably the dimethyl-C₁₇₋₃₂-alkyl sulfonium halide.

Typically, the insertion of a third alkyl group via reaction of a dialkyl sulfide and an alkyl halide is chemically demanding and the reactivity of alkyl halides decreases with increasing chain length. Therefore, yields of the obtained trialkyl sulfonium halides are also expected to decrease accordingly with increasing chain length of the alkyl halide. However, it has been surprisingly found that the process according to the invention provides dimethyl-C₁₇₋₃₂-alkyl sulfonium salts, preferably dimethyl-C₁₇₋₃₂-alkyl sulfonium halides, i.e. dimethyl-alkyl sulfonium halides, more preferably dimethyl-C₁₇₋₃₂-alkyl sulfonium chlorides, having an alkyl chain with a chain length within the range of from 17 to 32, preferably 18, in high yields at short reaction times.

It has been surprisingly found that the dimethyl-C₁₇₋₃₂-alkyl sulfonium salt, preferably the dimethyl-C₁₇₋₃₂-alkyl sulfonium halides, more preferably dimethyl-C₁₇₋₃₂-alkyl sulfonium chlorides, can be provided in higher yields when the mixture provided in step (c) of the process according to the invention comprises an acid. Reacting the C₁₇₋₃₂-alkyl halide and dimethyl sulfide in the presence of the acid preferably provides a product composition comprising a dimethyl-C₁₇₋₃₂-alkyl sulfonium acid salt and optionally remaining C₁₇₋₃₂-alkyl halide, acid and/or dimethyl sulfide. The obtained dimethyl-C₁₇₋₃₂-alkyl sulfonium acid salt may then be converted into the dimethyl-C₁₇₋₃₂-alkyl sulfonium salt, preferably the dimethyl-C₁₇₋₃₂-alkyl sulfonium halide. Thus, the dimethyl-C₁₇₋₃₂-alkyl sulfonium salt is preferably not obtained directly by the reaction of the C₁₇₋₃₂-alkyl halide and the dimethyl sulfide, but by conversion of the dimethyl-C₁₇₋₃₂-alkyl sulfonium acid salt. Further, it has been surprisingly found that separating at least the majority of the dimethyl-C₁₇₋₃₂-alkyl sulfonium acid salt from the product composition prior to conversion into the dimethyl-C₁₇₋₃₂-alkyl sulfonium salt, preferably the dimethyl-C₁₇₋₃₂-alkyl sulfonium halide further improves the yield and additionally increases the purity of the dimethyl-C₁₇₋₃₂-alkyl sulfonium salt, preferably the dimethyl-C₁₇₋₃₂-alkyl sulfonium halide obtained. When the C₁₇₋₃₂-alkyl halide and the dimethyl sulfide are reacted directly to give the dimethyl-C₁₇₋₃₂-alkyl sulfonium salt, preferably the dimethyl-C₁₇₋₃₂-alkyl sulfonium halide, i.e. without separating the dimethyl-C₁₇₋₃₂-alkyl sulfonium acid salt prior to conversion into the dimethyl-C₁₇₋₃₂-alkyl sulfonium salt, preferably the dimethyl-C₁₇₋₃₂-alkyl sulfonium halide, the obtained product may be a mixture of the dimethyl-C₁₇₋₃₂-alkyl sulfonium acid salt and the dimethyl-C₁₇₋₃₂-alkyl sulfonium salt, preferably the dimethyl-C₁₇₋₃₂-alkyl sulfonium halide. Thus, the overall yield and purity of the dimethyl-C₁₇₋₃₂-alkyl sulfonium salt, preferably the dimethyl-C₁₇₋₃₂-alkyl sulfonium halide may be significantly reduced without the additional separation step.

Another aspect of the invention relates to a process for the preparation of an agricultural composition comprising a dimethyl-C₁₇₋₃₂-alkyl sulfonium salt, preferably dimethyl-C₁₇₋₃₂-alkyl sulfonium halide, wherein the process for the preparation of the agricultural composition comprises the process for the preparation of a dimethyl-C₁₇₋₃₂-alkyl sulfonium salt, preferably dimethyl-C₁₇₋₃₂-alkyl sulfonium halide, according to the invention as describe above.

Preferably, the agricultural composition is selected from solutions, suspensions, emulsions, gels, mousses, pastes, powders and granules; preferably a liquid or a paste; or a solid.

Preferably, the content of the dimethyl-C₁₇₋₃₂-alkyl sulfonium salt, preferably the dimethyl-C₁₇₋₃₂-alkyl sulfonium halide, is at least 0.5 wt.-%, preferably at least 1.0 wt.-%, preferably at least 2.5 wt. %, preferably at least 5 wt.-%, preferably at least 7.5 wt.-%, preferably at least 10 wt.-%, preferably at least 12.5 wt.-%, preferably at least 15 wt.-%, preferably at least 17.5 wt.-%, preferably at least 20 wt. %, in each case relative to the total weight of the agricultural composition.

Preferably, the content of the dimethyl-C₁₇₋₃₂-alkyl sulfonium salt, preferably the dimethyl-C₁₇₋₃₂-alkyl sulfonium halide, is at most 97.5 wt.-%, preferably at most 95 wt.-%, preferably at most 92.5 wt.-%, preferably at most 90 wt.-%, preferably at most 87.5 wt.-%, preferably at most 85 wt.-%, preferably at most 82.5 wt.-%, preferably at most 80 wt.-%, in each case relative to the total weight of the agricultural composition.

Preferably, the content of the dimethyl-C₁₇₋₃₂-alkyl sulfonium salt, preferably the dimethyl-C₁₇₋₃₂-alkyl sulfonium halide, is within the range of from 10 to 80 wt.-%, relative to the total weight of the agricultural composition.

In preferred embodiments, the content of the dimethyl-C₁₇₋₃₂-alkyl sulfonium salt, preferably the dimethyl-C₁₇₋₃₂-alkyl sulfonium halide, is
at least 2.5 μg/g; preferably at least 5.0 μg/g, preferably at least 7.5 μg/g, preferably at least 10 μg/g, preferably at least 15 μg/g, preferably at least 20 μg/g, preferably at least 25 μg/g, preferably at least 30 μg/g, preferably at least 40 μg/g, preferably at least 50 μg/g, preferably at least 60 μg/g, preferably at least 70 μg/g, preferably at least 80 μg/g, preferably at least 90 μg/g; and/or
at most 200 μg/g, preferably at most 190 μg/g, preferably at most 180 μg/g, preferably at most 170 μg/g, preferably at most 160 μg/g, preferably at most 150 μg/g, preferably at most 140 μg/g, preferably at most 130 μg/g, preferably at most 120 μg/g, preferably at most 110 μg/g, preferably at most 100 μg/g, preferably at most 90 μg/g, preferably at most 80 μg/g, preferably at most 70 μg/g, preferably at most 60 μg/g, preferably at most 50 μg/g, preferably at most 40 μg/g, preferably at most 30 μg/g, preferably at most 20 μg/g, preferably at most 10 μg/g; in each case relative to the total weight of the agricultural composition.

Preferably, the process comprises the step of
(e) mixing the dimethyl-C₁₇₋₃₂-alkyl sulfonium salt, preferably the dimethyl-C₁₇₋₃₂-alkyl sulfonium halide, with
   an agriculturally acceptable carrier;
   optionally, with one or more additives independently of one another selected from pH buffering agents, thickening agents, deposition agents, water conditioning agents, wetting agents, humectants, leaf cuticle and/or cell membrane penetration aids, surfactants, plant growth enhancers, foaming agents, defoaming agents, spreading agents, drift control agents, spray drift reducing agents, evaporation reducing agents, dyes, and UV absorbents; and/or
   optionally, with one or more further antifungal agents.

In preferred embodiments, the content of the carrier is at least 1.0 wt.-%, relative to the total weight of the agricultural composition; preferably at least 2.5 wt.-%; preferably at least 5.0 wt.-%, preferably at least 7.5 wt.-%, preferably at least 10 wt.-%, preferably at least 15 wt.-%, preferably at least 20 wt.-%, preferably at least 25 wt.-%, preferably at least 30 wt.-%, preferably at least 40 wt.-%, preferably at least 50 wt.-%, preferably at least 60 wt.-%, preferably at least 70 wt.-%, preferably at least 80 wt.-%, preferably at least 90 wt.-%; in each case relative to the total weight of the composition.

In preferred embodiments, the carrier is a solvent; preferably wherein the dimethyl-C₁₇₋₃₂-alkyl sulfonium salt, preferably the dimethyl-C₁₇₋₃₂-alkyl sulfonium halide, is completely dissolved in the carrier.

Preferably, the carrier is or comprises a constituent selected from the group consisting of (a) water; (b) monoalcohols such as methanol, ethanol, propanol, isopropanol, cyclohexanol, or benzyl alcohol; (c) glycols such as ethylene glycol, propylene glycol, diethylene glycol, or dipropylene glycol; (d) monoalkyl glycol ethers such as triethylene glycol monobutyl ether; (e) dialkyl glycol ethers such as ethylene glycol dimethylether; (f) glycol esters; (g) glycerol and glycerol ethers such as isopropylidine glycerol; (h) cyclic ethers such as tetrahydrofuran or dioxolane; (i) ketones such as acetone, butanone, or cyclohexanone; (j) monobasic esters such as ethyl lactate, ethyl acetate, or gamma-butyrolactone; (k) dibasic esters such as glutaric acid dimethylester or succinic acid dimethylester; (1) alkylene carbonates such as ethylene carbonate or propylene carbonate; (m) dialkyl sulfoxides such as dimethyl sulfoxide; (n) alkylsulfones such as sulfolanes; (o) alkyl amides such as N-methylpyrrolidone, N-ethylpyrrolidone, or dimethylformamide (p) alkanolamines such as monoethanolamine, diethanolamine, triethanolamine, alkyldiethanolamines, or dialkylmonoethanolamines; (q) fatty acids, fatty acid esters, fatty acid amides;
(r) oils such as oils of vegetable or animal origin, phytobland oils, crop oils, crop oil concentrates, vegetable oils, methylated seed oils, petroleum oils, and silicone oils; and combinations thereof. In particularly preferred embodiments, the carrier is or comprises water.

Preferably, the agricultural composition is aqueous and has a pH value within the range of from 2 to 14; preferably 3 to 13.

In preferred embodiments, the agricultural composition is aqueous and has a pH value of
at least 2.5, preferably at least 3.0, preferably at least 3.5, preferably at least 4.0, preferably at least 4.5, preferably at least 5.0, preferably at least 5.5, preferably at least 6.0, preferably at least 6.5, preferably at least 7.0, preferably at least 7.5, preferably at least 8.0, preferably at least 8.5, preferably at least 9.0, preferably at least 9.5, preferably at least 10, preferably at least 10.5, preferably at least 11;
at most 14, preferably at most 13.5, preferably at most 13, preferably at most 12.5, preferably at most 12, preferably at most 11.5, preferably at most 11.0, preferably at most 11.5, preferably at most 11.0, preferably at most 10.5, preferably at most 10.0, preferably at most 9.5, preferably at most 9.0, preferably at most 8.5, preferably at most 8.0, preferably at most 7.5; and/or
within the range of 3.0±2.0, preferably 3.0±1.0; or within the range of 4.0±3.0, preferably 4.0±2.0, preferably 4.0±1.0; or within the range of 5.0±4.0, preferably 5.0±3.0, preferably 5.0±2.0, preferably 5.0±1.0; or within the range of 6.0±5.0, preferably 6.0±4.0, preferably 6.0±3.0, preferably 6.0±2.0, preferably 6.0±1.0; or within the range of 7.0±6.0, preferably 7.0±5.0, preferably 7.0±4.0, preferably 7.0±3.0, preferably 7.0±2.0, preferably 7.0±1.0; or within the range of 8.0±5.0, preferably 8.0±4.0, preferably 8.0±3.0, preferably 8.0±2.0, preferably 8.0±1.0; or within the range of 9.0±4.0, preferably 9.0±3.0, preferably 9.0±2.0, preferably 9.0±1.0; or within the range of 10±4.0, preferably 10±3.0, preferably 10±2.0, preferably 10±1.0; or within the range of 11±3.0, preferably 11±2.0, preferably 11±1.0; within the range of 12±2.0, preferably 12±1.0.

Preferably, the carrier is or comprises a constituent selected from the group consisting of (a) natural soil minerals and mineral earth, such as silicates, calcites, marble, pumice, sepiolite, talc, kaolins, clays, talc, limestone, lime, calcium carbonate, chalk, bole, loess, quartz, perlite, attapulgite, montmorillonite, vermiculite, bentonite, dolomite, or diatomaceous earths; (b) synthetic minerals, such as silica, silica gels, alumina or silicates, such as aluminum silicates or magnesium silicates; (c) inorganic salts, such as aluminum sulfate, calcium sulfate, copper sulfate, iron sulfate, magnesium sulfate, silicon sulfate, magnesium oxide; (d) synthetic granules of inorganic or organic flours; (e) granules of organic material such as sawdust, coconut shell, corn ear or envelope, or tobacco stem; (f) kieselguhr; (g) tricalcium phosphate; (h) polysaccharides, such as cellulose, cellulose ethers, starch, xanthan, pullulan, guar; (i) products of vegetable origin, e.g. cereal meal, tree bark meal, wood meal, nutshell meal; (j) grain flours such as flours from corn, rice, wheat, barley, sorghum, millet, oat, triticale, rye, buckwheat, fonio or quinoa; (k) other organic matter such as powdered cork, adsorbent carbon black, charcoal, peat, soil mixture, compost, agro-industrial residues; water-soluble polymers, resins or waxes; (1) solid fertilizers such as urea or ammonium salts such as ammonium sulfate, ammonium phosphate, ammonium nitrate; and combinations thereof.

In preferred embodiments, the one or more further antifungal agents are independently of one another selected from (1) inhibitors of the ergosterol synthesis; (2) inhibitors of the respiratory chain at complex I or II; (3) inhibitors of the respiratory chain at complex III; (4) inhibitors of the mitosis and cell division; (5) compounds capable of having a multisite action; (6) compounds capable of inducing a host defense; (7) inhibitors of the amino acid and/or protein biosynthesis; (8) inhibitors of the ATP production; (9) inhibitors of the cell wall synthesis; (10) inhibitors of the lipid and membrane synthesis; (11) inhibitors of the melanine biosynthesis; (12) inhibitors of the nucleic acid synthesis; (13) inhibitors of the signal transduction; (14) compounds capable of acting as uncoupler; and (15) other fungicides.

Preferably, the one or more further antifungal agents are independently of one another selected from azoles; amino-derivatives; strobilurins; specific anti-oidium compounds; aniline-pyrimidines; benzimidazoles and analogues; dicarboximides; polyhalogenated fungicides; systemic acquired resistance inducers; phenylpyrroles; acylalanines; anti-peronosporic compounds; dithiocarbamates; arylamidines; phosphorous acid and its derivatives; fungicidal copper compounds; plant-based oils (botanicals); chitosan; sulfur-based fungicides; fungicidal amides; and nitrogen heterocycles.

Another aspect of the inventio relates to a dimethyl-C₁₇₋₃₂-alkyl sulfonium acid salt having general formula (V):

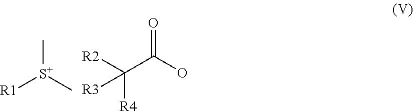

wherein
R1 represents —(CH₂)˜—CH₃, wherein n is an integer within the range of from 16 to 31; and
wherein R2, R3 and R4 independently of one another represent —H, —Cl, or —F; with the proviso that at least one of R2, R3 and R4 does not represent —H.

Preferably, R1 in general formula (V) is —(CH₂)₁₇—CH₃, and R2, R3 and R4 in general formula (V) are —F.

Preferably, the dimethyl-C₁₇₋₃₂-alkyl sulfonium acid salt according to the invention is dimethyloctadecyl sulfonium trifluoroacetate.

The following examples illustrate the invention and are not to be construed as limiting its scope.

Synthesis of dimethyl(octadecyl)sulfonium chloride 5:

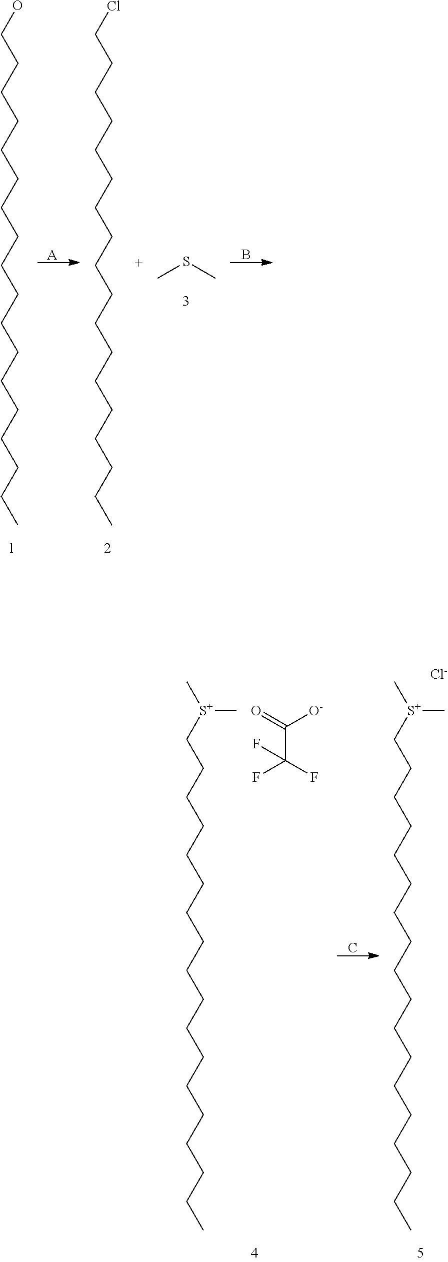

### Step A—Example A-3

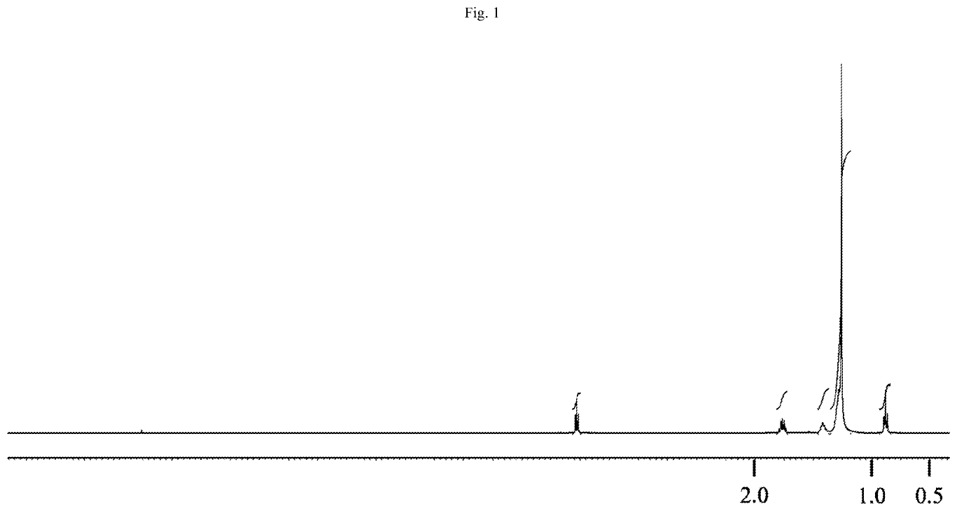

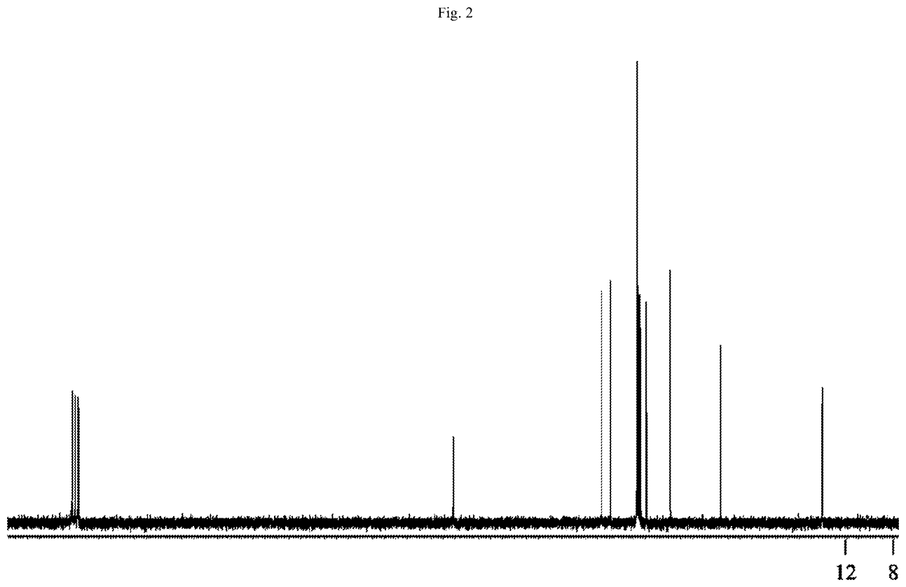

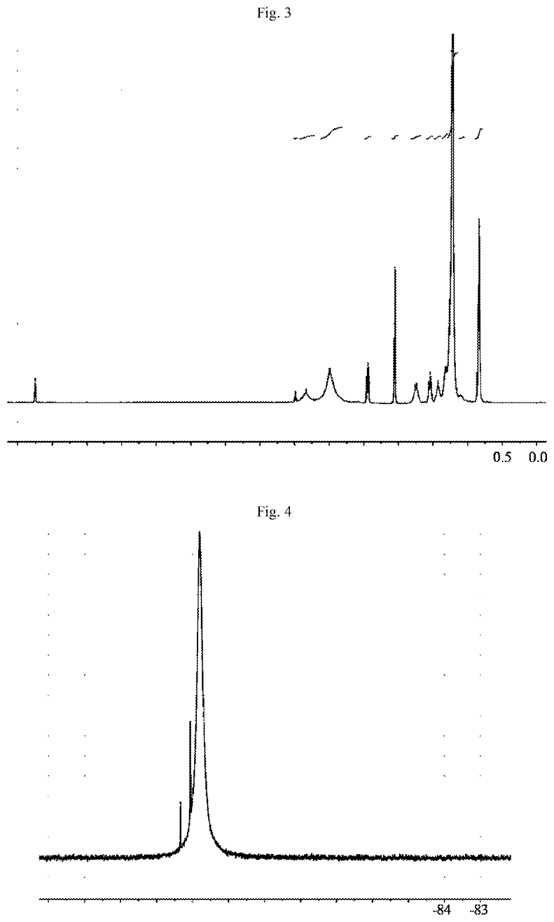

In 6 L glassware the alcohol 1 (500 g, 1.85 mol, 14eq was dissolved in anhydrous DCM (4 L, stock compound) and DMF (50 mL, stock compound) was added. SOCl₂ (˜400 mL, 5.55 mol, 3 eq, stock compound) was added dropwise at RT and was then left at 50° C. for 24 h after concentrated under reduced pressure (˜15 mm Hg) at 60° C., then dried from SOCl₂ residues under reduced pressure (˜1 mmHg) at 60° C. dissolved in 3 L hexane: methyl tert-butyl ether (MTBE)=9:1 and filtered through silica. Finally, pure material dried under reduced pressure (˜1 mm Hg) at 50° C.—light yellow oil liquid 2˜440 g, ˜82% yield, 95% pure by ¹HNMR and ¹³CNMR (see FIG. 1 and FIG. 2).

### Step B—Example B-6

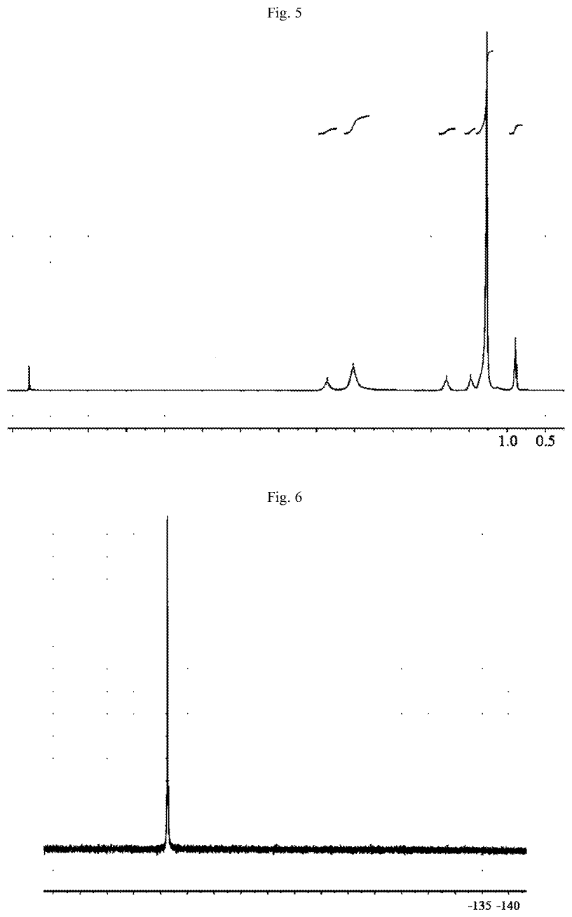

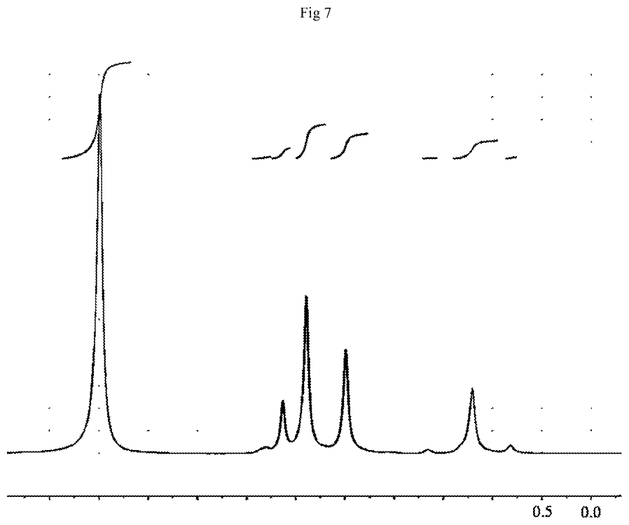

In Hastelloy (C-276) autoclave (1.2 L) to a solution of 2 (200 g, 0.69 mol, 1 eq, stock compound) in trifluoroacetic acid (TFA) (700 ml, stock compound) dimethyl sulfide, 3 (65 g 1.035 mol, 1.5 eq, stock compound) was added. The mixture was stirred at 110° C. for 24 h, after filtered and washed by MTBE (˜1 L), filtrate concentrated under reduced pressure (A15 mm Hg) at 60° C. and dried from CF₃COOH residues under reduced pressure (˜1 mmHg) at 50° C. Crude material was dissolved in mixture of diethyl ether (˜1 L, stock compound) and pentane (˜1 L, stock compound) at 40° C. after filtered and filtrate cooled to −50° C., then filtered and washed by cold mixture Et₂O: pentane=1:1 one time (A1 L, stock compounds) and three times by pentane (A1 L x3, stock compound). 135 g of residue remained, the ¹HNMR and ¹⁹FNMR are shown in FIG. 3 and FIG. 4. Pure material dried under reduced pressure (A1 mm Hg) at 50° C.—grey crystals 4 216 g—72%, 100% pure by ¹HNMR, ¹⁹FNMR (see FIG. 5 and FIG. 6).

### Step C—Example C-4

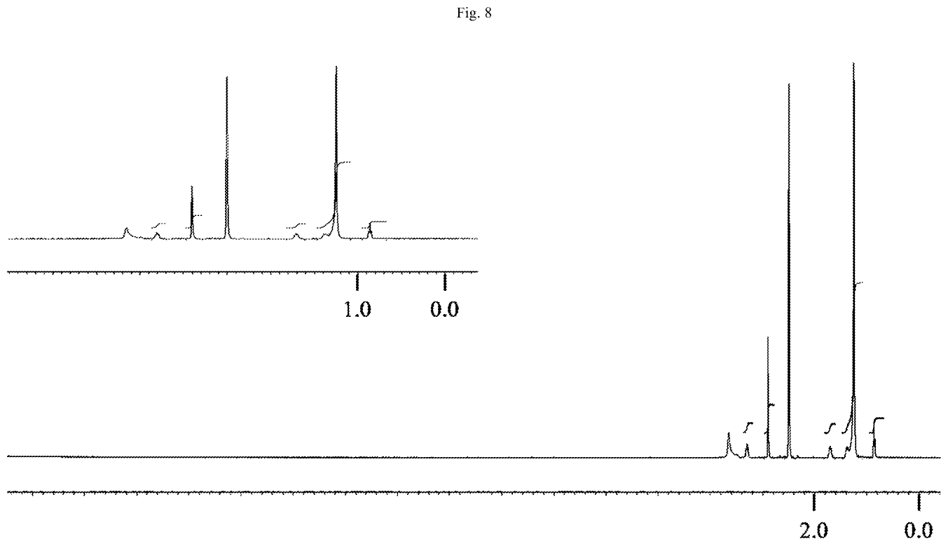

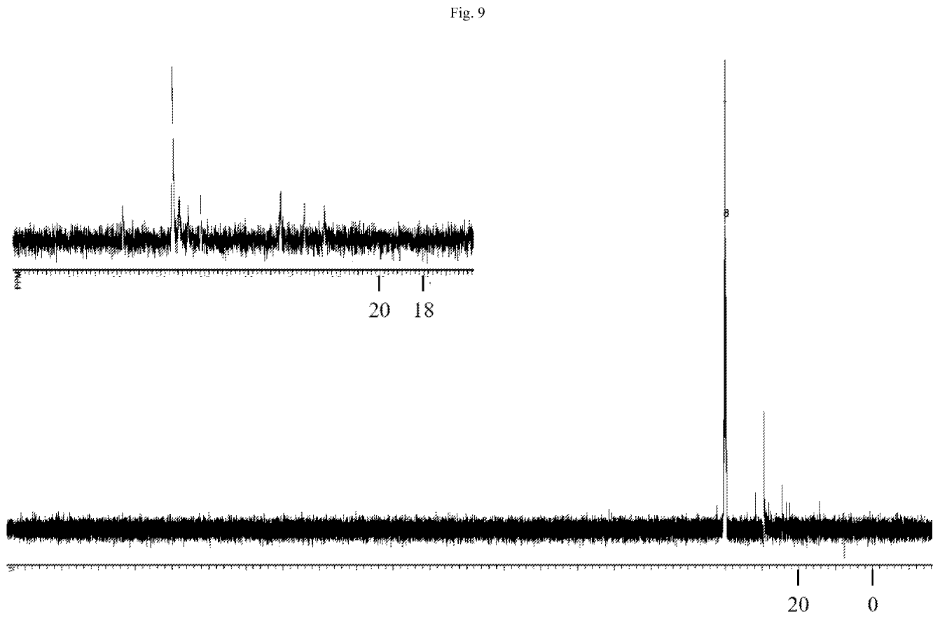

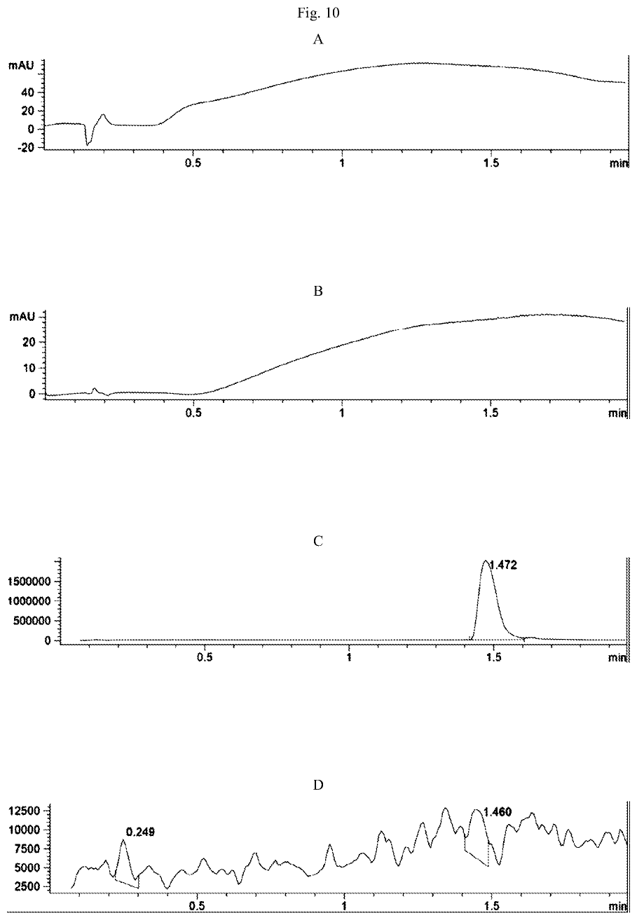

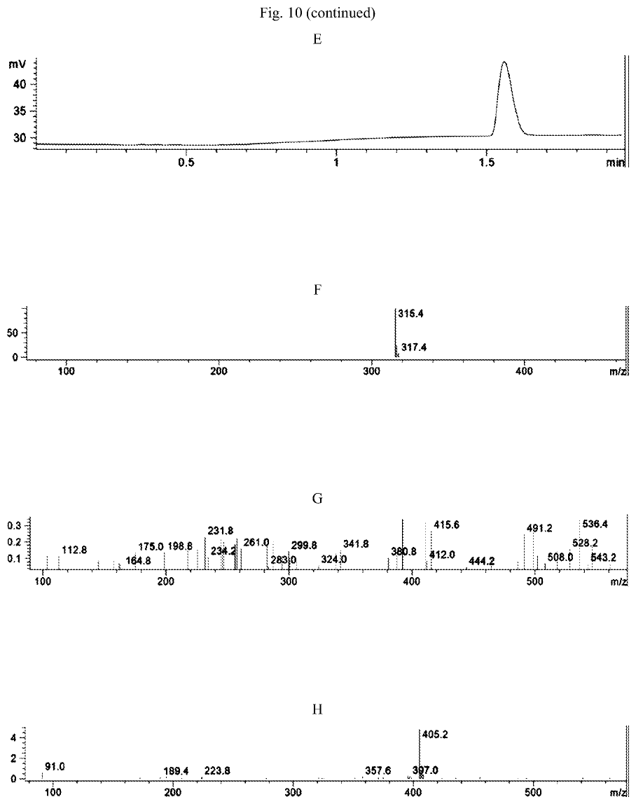

4 (216 g, 0.5 mol, 100% pure by ¹HNMR (see FIG. 6)) was dissolved in freshly prepared HCl (˜10 e) in MeOH (˜2 L, stock compound in 4 L glassware) SOCl₂ (˜200 mL, stock compound) was added dropwise at −10° C. and left for 15 hours at 40° C. Then concentrated under reduced pressure (˜15 mmHg) at 40° C., dried from MeOH residues under reduced pressure (˜1 mm Hg) at 40° C. Recrystallization in acetone (A1.5 L, stock compound). 45 g of residue remained, the ¹HNMR is shown in FIG. 7. Pure material dried under reduced pressure (A1 mm Hg) at RT—grey crystals 5 118 g—66%, 100% pure by ¹HNMR, ¹³CNMR and LCMS (see FIG. 8, FIG. 9, and FIG. 10A-H).

Calc. for (C₂₀H₄₃S)Cl: C, 68.42; H, 12.35; S, 9.13; Cl, 10.1. Found: C, 66.81; H, 12.24; S, 8.43; Cl, 11.39%.

### Chemical Analysis of 5:

LC-QToF Content of Active Ingredient: 1000 g/kg corresponding to 100 wt.-%
Moisture Content by Karl Fischer: 15.27 g/kg corresponding to 1.527 wt.-%
Trifluoro Acetic Acid by Ion Chromatography: <0.25 g/kg corresponding to <0.025 wt.-%
Alcohol Screening by GC-MS: <0.10 g/kg corresponding to <0.01 wt.-%
Solvent Screening by GC-MS: <0.01 g/kg corresponding to <0.001 wt.-%
Chloride by Ion Chromatography: 118.60 g/kg corresponding to 11.860 wt.-%
Theoretical Chloride Content: 99.49 g/kg corresponding to 9.949 wt.-%
Free Chlorine Content: 19.10 g/kg corresponding to 1.910 wt.-%
Authenticated Content of active Ingredient: 965.6 g/kg corresponding to 96.56 wt.-%.

### Step A

| | 1 | | SOCl₂ | | | Crude | Pure yield | |
|---|---|---|---|---|---|---|---|---|
| | | m | | V | | yield | | m |
| | eq. | [g] | eq. | [mL] | Conditions | [%] | [%] | [g] |
| A-1 | 1 | 1 | 186 | 50 | 80° C./~15 h | ~70 | n.d. | n.d. |
| A-2 | 1 | 30 | 37 | 300 | 50° C./~15 h | ~100 | 90 | 28 |
| A-3 | 1 | 500 | 3 | 400 | 50° C./~24 h | 100-110⁺ | 82 | 440 |

⁺The crude products were obtained by evaporation of the reaction mixture up to the weight of 100%-110% form the expected yields.

### Step B:

| | 2 | | 3 | | TFA | | | Crude | Pure yield | |
|---|---|---|---|---|---|---|---|---|---|---|
| | eq. | m [g] | eq. | m [g] | eq. | V [mL] | Conditions | yield [%] | [%] | m [g] |
| B-1 | 1 | 5 | 0.5 | 0.54 | 19 | 25 | 100° C./~20 h | 79* | 55%* | 1.4 |
| B-2 | 1 | 5 | 1 | 1.08 | 19 | 25 | 100° C./~20 h | 67 | 47% | 2.5 |
| B-3 | 1 | 5 | 1 | 1.08 | 1 | 1.3 | 100° C./~20 h | 7.5 | 2.5% | 0.1 |
| B-4 | 1 | 5 | 1.5 | 1.62 | 19 | 25 | 100° C./~20 h | 100 | 85% | 4.6 |
| B-5 | 1 | 20 | 1.5 | 6.5 | 19 | 100 | 110° C./~24 h | 100-110⁺ | 85 | 25 |
| B-6 | 1 | 200 | 1.5 | 65 | 13 | 700 | 110° C./~24 h | 100-110⁺ | 72 | 216 |

*The yield is based on 3.
⁺The crude products were obtained by evaporation of the reaction mixture up to the weight of 100%-110% form the expected yields.

### Synthesis of Dimethyl(Octadecyl)Sulfonium Bromide 6:

| | 4 | | SOCl₂ | | | Crude | Pure yield | |
|---|---|---|---|---|---|---|---|---|
| | eq. | m [g] | eq. | V [mL] | Conditions | yield [%] | [%] | m [g] |
| C-1 | 1 | 1 | 10 | ~2 | −20-rt° C./~24 h | 100 | 90 | ~0.7 |
| C-2 | 1 | 15 | 10 | ~30 | 50° C./~15 h | 100 | 95 | ~12 |
| C-3 | 1 | 25 | | | 40° C./~15 h | 100-110⁺ | 95 | 19 |
| C-4 | 1 | 216 | 5 | ~200 | 40° C./~15 h | 100-110⁺ | 66 | 118 |

⁺The crude products were obtained by evaporation of the reaction mixture up to the weight of 100%-110% form the expected yields.

### Step C—Example C-5

4 (10 g, 0.023 mol) was dissolved in MeOH and added aqueous HBr (47%, 10 eq, ˜40 ml) and left for 15 hours at 50° C. Then concentrated under reduced pressure, filtered, washed by diethyl ether and recrystallization in acetone. Yield 6 ˜7 g—80%.

### Synthesis of Dimethyl(Octadecyl)Sulfonium Iodide 7:

### Step C—Example C-6

4 (10 g, 0.023 mol) was dissolved in MeOH and added aqueous HI (57%, 10 eq, ˜50 ml) and left for 15 hours at 50° C. Then concentrated under reduced pressure, filtered, washed by diethyl ether and recrystallization in acetone. Yield 7 8 g—80%.

1. A process for the preparation of a dimethyl-C₁₇₋₃₂-alkyl sulfonium salt, comprising the steps of:
(c) providing a mixture of a C₁₇₋₃₂-alkyl halide and dimethyl sulfide; and
(d) reacting the C₁₇₋₃₂-alkyl halide with the dimethyl sulfide.

2. The process according to claim 1, which comprises the preceding steps of:
(a) providing a mixture of a C₁₇₋₃₂-alkyl alcohol and a halide donor; and
(b) reacting the C₁₇₋₃₂-alkyl alcohol with the halide donor thereby providing the C₁₇₋₃₂-alkyl halide.

3. The process according to claim 2, wherein the C₁₇₋₃₂-alkyl alcohol has general formula (I):
R1—OH (I),
wherein R1 represents —(CH₂)ₙ—CH₃, wherein n is an integer within the range of from 16 to 31.

4.-7. (canceled)

8. The process according to claim 1, wherein the C₁₇₋₃₂-alkyl halide has general formula (III)
R1—X (III)
wherein
R1 represents —(CH₂)ₙ—CH₃, wherein n is an integer within the range of from 16 to 31; and
X is selected from the group consisting of —F, —Cl, —Br, and —I.

9.-10. (canceled)

11. The process according to claim 1, wherein the mixture provided in step (c) additionally comprises an acid differing from HF, HCl, HBr and HI.

12.-15. (canceled)

16. The process according to claim 1, wherein step (c) comprises the sub-step of:
(c-1) providing a composition comprising the C₁₇₋₃₂-alkyl halide and a solvent.

17-20. (canceled)

21. The process according to claim 1, wherein the dimethyl sulfide and the C₁₇₋₃₂-alkyl halide are provided in step (c) in a molar ratio of
at least 0.52; and/or
within the range of 0.75±0.23, or 1.0±0.48, or 1.0±0.23, or 1.25±0.73, or 1.25±0.48, or 1.25±0.23, or 1.5±0.98, 1.5±0.73, or 1.5±0.48, or 1.5±0.23.

22. The process according to claim 11, wherein step (d) comprises the sub-step of:
(d-1) reacting the C₁₇₋₃₂-alkyl halide and dimethyl sulfide in the presence of the acid thereby providing a dimethyl-C₁₇₋₃₂-alkyl sulfonium acid salt.

23.-26. (canceled)

27. The process according to claim 22, wherein substep (d-1) is performed under elevated pressure.

28.-33. (canceled)

34. The process according to claim 22, wherein step (d) comprises the sub-step of:
(d-4) converting the dimethyl-C₁₇₋₃₂-alkyl sulfonium acid salt separated in sub-step (d-2) into the dimethyl-C₁₇₋₃₂-alkyl sulfonium salt.

35.-37. (canceled)

38. The process according to claim 1, wherein the dimethyl-C₁₇₋₃₂-alkyl sulfonium salt has general formula (VI) 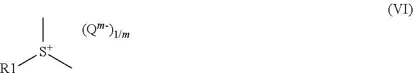
wherein
R1 represents —(CH₂)ₙ—CH₃, wherein n is an integer within the range of from 16 to 31;
Q is X or the conjugate base of the further acid, wherein X is selected from the group consisting of —F, —Cl, —Br, and —I; and m is an integer of 1, 2, or 3.

39.-42. (canceled)

43. The process according to claim 34, wherein substep (d-4) comprises
(i) dissolving the dimethyl-C₁₇₋₃₂-alkyl sulfonium acid salt in an acid and/or a solvent;
(ii) optionally, cooling the mixture thus obtained in step (i);
(iii) adding the halide donor as defined above to the mixture thus obtained in step (i) or step (ii);
(iv) optionally, heating the mixture thus obtained in step (iii);
(v) allowing the dimethyl-C₁₇₋₃₂-alkyl sulfonium acid salt and the halide donor to form the dimethyl-C₁₇₋₃₂-alkyl sulfonium salt;
(vi) removing of volatile compounds from the mixture thus obtained in step (v) under reduced pressure;
(vii) optionally, recrystallizing the residue thus obtained in step (vi) from an organic solvent; and
(viii) optionally, drying the residue thus obtained in step (vi) or step (vii) to obtain the dimethyl-C₁₇₋₃₂-alkyl sulfonium salt.

44. The process according to claim 34, wherein substep (d-4) comprises
(i) dissolving the dimethyl-C₁₇₋₃₂-alkyl sulfonium acid salt in the further acid and/or a solvent;
(ii) optionally, heating the mixture thus obtained in step (i);
(iii) allowing the dimethyl-C₁₇₋₃₂-alkyl sulfonium acid salt and the further acid to form the dimethyl-C₁₇₋₃₂-alkyl sulfonium salt;
(iv) concentrating the mixture thus obtained in step (iii);
(v) optionally, filtering the residue thus obtained in step (iv) and washing the feed thus obtained in step (v) with an organic solvent;
(vi) optionally, recrystallizing the residue thus obtained in step (v) from an organic solvent; and
(vii) optionally, drying the residue thus obtained in any of steps (iv) to (vi) to obtain the dimethyl-C₁₇₋₃₂-alkyl sulfonium salt.

45. The process according to claim 34, wherein substep (d-4) comprises
(i) dissolving the dimethyl-C₁₇₋₃₂-alkyl sulfonium acid salt in the further acid and/or a solvent;
(ii) optionally, heating the mixture thus obtained in step (i);
(iii) allowing the dimethyl-C₁₇₋₃₂-alkyl sulfonium acid salt and the further acid to form the dimethyl-C₁₇₋₃₂-alkyl sulfonium salt;
(iv) concentrating the mixture thus obtained in step (iii);
(v) optionally, filtering residue thus obtained in step (iv) and washing the feed thus obtained in step (v) with an organic solvent;
(vi) optionally, recrystallizing residue thus obtained in step (v) from an organic solvent; and
(vii) optionally, drying the residue thus obtained in any of steps (iv) to (vi) to obtain the dimethyl-C₁₇₋₃₂-alkyl sulfonium salt.

46. A process for the preparation of an agricultural composition comprising a dimethyl-C₁₇₋₃₂-alkyl sulfonium salt, wherein the process for the preparation of the agricultural composition comprises conducting the process for the preparation of a dimethyl-C₁₇₋₃₂-alkyl sulfonium salt according to claim 1.

47. (canceled)

48. The process according to claim 46, wherein the content of the dimethyl-C₁₇₋₃₂-alkyl sulfonium salt is at least 0.5 wt.-%, relative to the total weight of the agricultural composition.

49.-51. (canceled)

52. The process according to claim 46, which comprises the step of (e) mixing the dimethyl-C₁₇₋₃₂-alkyl sulfonium salt with
an agriculturally acceptable carrier;
optionally, with one or more additives independently of one another selected from pH buffering agents, thickening agents, deposition agents, water conditioning agents, wetting agents, humectants, leaf cuticle and/or cell membrane penetration aids, surfactants, plant growth enhancers, foaming agents, defoaming agents, spreading agents, drift control agents, spray drift reducing agents, evaporation reducing agents, dyes, and UV absorbents; and/or
optionally, with one or more further antifungal agents.

53.-56. (canceled)

57. The process according to claim 52, wherein the agricultural composition is aqueous and has a pH value within the range of from 2 to 14.

58.-61. (canceled)

62. A dimethyl-C₁₇₋₃₂-alkyl sulfonium acid salt having general formula (V): 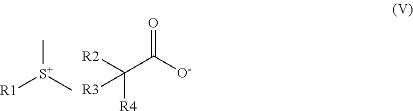
wherein
R1 represents —(CH₂)ₙ—CH₃, wherein n is an integer within the range of from 16 to 31; and
R2, R3 and R4 independently of one another represent —H, —Cl, or —F; with the proviso that at least one of R2, R3 and R4 does not represent —H.

63. The dimethyl-C₁₇₋₃₂-alkyl sulfonium acid salt according to claim 62, wherein R1 is —(CH₂)₁₇—CH₃, and R2, R3 and R4 are —F.

64. The dimethyl-C₁₇₋₃₂-alkyl sulfonium acid salt according to claim 62, which is dimethyl-octadecyl sulfonium trifluoroacetate.
